(19) United States
(12) Patent Application Publication
(10) Pub. No.: US 2007/0218496 A1
(43) Pub. Date: Sep. 20, 2007

(54) METHOD FOR QUICK DETERMINATION OF CYTOKERATIN 19 (CK19) AND PRIMERS AND PROBES THEREFORE

(75) Inventors: Yuko Kitagawa, Tokyo (JP); Yasuhiko Sakakura, Yamato City (JP)

Correspondence Address: ROCHE MOLECULAR SYSTEMS INC;PATENT LAW DEPARTMENT, 1145 ATLANTIC AVENUE, ALAMEDA, CA, 94501, US

(73) Assignee: ROCHE MOLECULAR SYSTEMS, INC., Alameda, CA (US)

(21) Appl. No.: 11/734,099

(22) Filed: Apr. 11, 2007

(30) Foreign Application Priority Data
Mar. 11, 2006 (JP) 2006-108744

(51) Int. Cl. C12Q 1/68; C12P 19/34; C07H 21/04

(52) U.S. Cl. 435/6; 435/91.2; 536/25.32

(57) ABSTRACT

A method for determination of CK19 mRNA is provided characterized in that a part of CK19 mRNA is amplified using a first primer hybridizing to a region located on a first exon of the CK19 gene and a second primer hybridizing to a region located on a second exon of the CK19 gene locating downstream of the first exon. The amplificate is than detected using two kinds of probes.

### CROSS-REFERENCES TO RELATED APPLICATIONS

This application claims the benefit of priority under 35 U.S.C. §119 Japanese Patent Application No. 2006-108744, filed Apr. 11, 2006, the disclosure of which is incorporated by reference in its entirety for all purposes.

### FIELD OF THE INVENTION

The present invention relates to methods for the determination of CK19 mRNA, and for the determination of CK19 and CK20 mRNA, particularly in a clinical setting. Furthermore, probes and PCR primers as well as kits used for quick and specific amplification and detection of mRNA of CK19 on a real-time basis are provided.

### BACKGROUND OF THE INVENTION

In surgical operations, it is necessary to excise organs and tissues to which cancer metastasizes such as a lymph node in addition to the primary cancer. However, as mentioned herein above already, it is necessary to completely remove the contaminating DNA, etc. and to highly purify the aimed mRNA for the purpose of a precise detection of the expression (mRNA) of a specific gene related to cancer and, therefore, a long time is needed for judging the presence or absence of mRNA. On the other hand, there is a limit for the time allowed for a surgical operation. Accordingly, it has not been possible to judge during the operation whether mRNA specific to cancer is present in tissues and organs collected in an operation. Therefore, in order to prevent the recurrence of metastasis of cancer after the operation, it has been necessary to widely excise the tissues and organs to which metastasis is possible such as the lymph nodes without judging whether cancer cells actually had metastasized.

With regard to the above, the burden for a patient is greatly reduced if it is possible to judge within a short time whether an mRNA of a gene related to a specific cancer is present in a sample of tissues and organs collected in a surgical operation. In this case only the tissues and organs in which the mRNA of a cancer-related gene is detected may be excised.

Cytokeratin is a group of proteins called an intermediate-sized filament forming cytoskeleton and, up to now, not less than 20 kinds of subspecies have been reported. Those cytokeratins are mostly present in epithelial cells and distribution of each subspecies is different depending upon the type of epithelial cells.

Cytokeratin 19 (CK19) has been reported to be expressed more abundantly in cancer tissues such as breast cancer, stomach cancer, prostate cancer and lung cancer than in normal tissues and has been investigated for its application to clinical diagnosis as a clinical marker for diagnosis of cancer and also as an index for selecting a therapeutic strategy. For example, in breast cancer in early stage or in stomach cancer in early stage, there is a possibility that the appropriateness for reducing the range for lymph node dissection during a surgical operation for improvement of quality of life (QOL) of a patient may be judged by diagnosis for metastasis of cancer in lymph node using CK19 as an index. There may be also a possibility that, when the presence or absence of cancer cells in a washing of peritoneum is checked using CK19 as an index, the risk of a peritoneal seedling of a patient suffering from progressive stomach cancer may be evaluated. With regard to a method for detection a gene in high sensitivity, a LAMP method, a TRC method, a PCR method, etc. have been reported up to now.

A LAMP (loop mediated isothermal amplification) is a method where a target gene is amplified efficiently at predetermined temperature using four kinds of primers and DNA polymerase capable of strand displacement (WO 00/28082).

A TRC (transcription reverse transcription concerted reaction) is a method where RNA is amplified at a predetermined temperature (EP 0969101) where a target RNA region is amplified by means of trimming of a target RNA using an RNase activity of a reverse transcriptase and DNA oligonucleotide for cleaving of target RNA called a scissor probe and by means of repetition of reverse transcription reaction and transcription reaction. In this method, RNA is able to be directly amplified and, therefore, a reverse transcription step which is necessary in PCR and LAMP method is not necessary.

Steps for amplification are complicated in those amplifying methods for nucleic acid as compared with a PCR method and, in addition, it is not possible to amplify plural targets at the same time and to detect each of them in one reaction tube. This means that an internal control for discrimination of false negatives can not be integrated in a reaction system. Accordingly, such methods have the problem that, when applied to clinical diagnosis, false negative samples may not be discriminated.

A PCR (polymerase chain reaction) is a method where any DNA region may be amplified efficiently with good reproducibility. It has been widely used throughout the world from fundamental studies to practical industries in the fields of medicine, medical jurisprudence, veterinary science, agricultural science, pharmaceutical science, biology, etc. When this method is combined with an enzyme having a reverse transcription activity, it is also possible to amplify any RNA region (RT-PCR). Many means for detection of amplified products have been reported as well. When a fluorescent probe comprising a fluorescent dye bonded to a DNA oligonucleotide is added to a PCR solution, monitoring of the reaction may be carried out on a real-time basis (real-time PCR). It is also possible that using a plurality of fluorescent probes, each separately labeled with a different fluorescent dye, plural target DNAs (or RNAs) can be amplified at the same time and the pattern of such an amplifying reaction of each target nucleic acid may also be separately monitored (multiplex real-time PCR).

Although amplification of mRNA by an RT-PCR method is highly sensitive, the gene (DNA) which is a template for the mRNA is also amplified if human DNA is present in the sample to be tested, even if present only in a small amount. Accordingly, it may be difficult to judge whether the amplified product is derived from RNA or DNA.

In such methods there is a certain possibility for “false positive” results, in which the result which is to be negative is judged to be positive. This is a big problem when such methods are applied to clinical diagnosis.

As a means for avoiding such a problem, a design is carried out in amplification of RNA by an RT-PCR so that an intron comes into the region of the gene corresponding to such an RNA region.

In general, a gene is divided by an intron, which is a region having no genetic information. Accordingly, if an intron is present in a region which is amplified by RT-PCR, the PCR product where DNA was amplified is larger to the extent of the intron compared to the amplified product of mRNA. In that case, even when DNA is contaminating an mRNA sample to be subjected to PCR, it is possible to judge whether mRNA is amplified or DNA is amplified by means of an agarose gel electrophoresis or the like where the difference in size of the amplified product may be detected.

However, when a gene region where no intron is present is amplified, the size of the amplified product is the same as that in case of an mRNA. In this case it is not possible to distinguish a false positive result by electrophoresis.

When a human gene is contaminating a clinical sample, there is another problem where the DNA is a process-type pseudogene. A pseudogene does not function as a gene and, since no intron is present in a base sequence of a process-type pseudogene and since there is a high homology in view of base sequence to mRNA, it is not possible to discern a false positive result by way of the size of the amplified product.

The presence of process-type pseudogene in CK19 has been reported. The homology of the pseudogene to CK19 mRNA is not less than 90%. Accordingly, if amplification of the pseudogene takes place, it may not be possible to know whether the amplified product is derived from mRNA or derived from the pseudogene, comparable to the case of PCR of a gene region having no intron.

Thus, in case where CK19 mRNA is detected by a nucleic acid amplification method such as RT-PCR to be used in a clinical test, DNA which is contaminating the sample should be thoroughly excluded. In other words, when a positive result is interpreted, its application to a clinical test is not possible unless it may be discriminated whether the positive result is “true positive” due to CK19 mRNA or is “false positive” due to CK19 gene or CK19 pseudogene.

An effective means for preventing the generation of such a “false positive” is to thoroughly remove human DNA from the sample. However, in conventional methods, too much attention is paid on the degree of purification and, as a result, the time necessary for preparing the sample is long, a special apparatus is necessary and numbers of the samples which may be treated are limited. Therefore, the method is not always effective in the clinical field where speed and simplicity are demanded.

There have been many publications reporting PCR primers and detection probes for amplification and detection of CK19 mRNA by means of RT-PCR and there also have been reports mentioning that a special device is used not to amplify or detect the CK19 pseudogene (Yuan, C C., et al., Gynecol. Oncol., 85: (1) 148-153, 2002; Dimmler, A. et al., Laboratory Investigation, 81: (10) 1351-1361, 2000; Gazzaniga, P., et al., Clin. Cancer Res., 7: 577-583, 2001; Stathopoulou, A., et al., Clin. Cancer Res., 9: 5145-5151, 2003; Fellowes, V., et al., Int. J. Oncol., 24: 861-867, 2004). However, none of these publications provides the quickness which is demanded in the clinical field at present.

For example, a nested PCR is conducted in Yuan et al. However, in this method a time of 5 hours or longer is required in the PCR only. Particularly in Yuan et al. and in Stathopoulou et al. an agarose gel electrophoresis is used for detection of the amplified product whereby the judgment of the result of the PCR requires a greater amount of times. In addition, in preparing the sample for the PCR, all of these prior art methods (Yuan, C C., et al.; Dimmler, A. et al.; Gazzaniga, P., et al.; Stathopoulou, A., et al.; Fellowes, V.) carry out the PCR and the reverse transcription reaction of mRNA in separate reactions (two-step RT-PCR) and about one hour is necessary for the reverse transcription step only.

In Dimmler et al. and Fellowes et al. it is mentioned that the purified RNA sample is to be further subjected to a DNase treatment. In the practical medical field where a nucleic acid amplification method is used for clinical diagnosis, pollution with nuclease is not preferred. That is because, if pollution by DNase or RNase occurs in an operation chamber or in experimental instruments, there is a possibility of affecting the result of the gene test. Accordingly, with regard to a method for preparing a sample for nucleic acid amplification to be used in a clinical test, a method where no nuclease is used usually is preferred.

### BRIEF DESCRIPTION OF THE DRAWINGS

FIG. 1 is a graph which shows the result where CK19 mRNA which was diluted in a stepwise manner was subjected to the one-step RT-PCR of the present invention.

FIG. 2 is a graph which shows the result where human DNA and CK19 pseudogene were subjected to the same one-step RT-PCR as in the case of FIG. 1.

FIG. 3 is a graph which shows the result where lymph nodes (#1 to #2) in which metastasis of cancer was positive by a pathological diagnosis and lymph node (#3) in which metastasis of cancer was negative by a pathological diagnosis were subjected to the one-step RT-PCR of the present invention.

### SUMMARY OF THE INVENTION

The present invention provides a method to determine whether the mRNA of CK19 is present in a sample of tissue and/or organ collected in a surgical operation and/or a method to determine whether said tissues and organs are to be excised within such a short time that the facts may be evaluated during the operation.

The present invention resolves the aforementioned problem by the selection of appropriate primers and probes. Thus, the present invention discloses a combination of PCR primers and detection probes by which amplification of the pseudogene is prevented by the selectivity of the PCR primers and, further, the gene is not detected by the selection of the location of the detection probes. When the primers and probes of the present invention are used, there is no necessity of treating a sample for which a PCR is conducted with DNase. Moreover, as will be shown in the Examples, when a nucleic acid synthesizing element having reverse transcription ability is used, the time required for amplification and detection of the CK19 mRNA may be shortened to about 40 minutes by conducting the reverse transcription step and the PCR step in the same reactor and also by conducting a real-time PCR.

Accordingly, the present invention provides a method for the determination of CK19 mRNA, comprising
(a) amplifying a part of the mRNA in said sample using a pair of primers, to produce an amplicon, and
(b) detecting the amplicon using two probes;
   wherein the primers comprise, at least
a first primer capable of hybridizing to a 1^{st} region located on a first exon of the CK19 gene, and
a second primer capable of hybridizing to a 2^{nd} region located on a second exon of the CK19 gene located downstream from the first exon; and
   wherein the probes comprise, at least
a first probe capable of hybridizing to a 3^{rd} region located at the 3′-terminus of a first exon of the CK19 gene, said region being located downstream of the region to which the first primer binds and upstream of the region to which the second primer binds, and
   a second probe capable of hybridizing to a 4^{th} region located at the 5′-terminus of a second exon of the CK19 gene, said second exon being adjacent to the 1^{st} exon and being downstream to the 4^{th} region to which the first probe binds and upstream to the region to which the second primer binds.

In the above-mentioned method, the probes in certain embodiments may be labeled with a donor dye and an acceptor dye. For example, the above donor dye may be a fluorescein dye, such as FITC, and the acceptor dye may be selected from the group consisting of rhodamine dyes and cyanine dyes. The first probe may in certain embodiments be labeled with a donor dye at its 3′-terminus and the second probe may be labeled with an acceptor dye at its 5′-terminus. As a specific example, the donor dye is FITC and the acceptor dye is LC-Red 640, which is a rhodamine dye.

Rhodamine dyes are derived from the fusion of phthalic anhydride with m-dialkylaminophenol. Such dyes are disclosed, for example, in EP 567,622 and it is mentioned that pyrano[3,2-g:5,6-g′] diguanoline-13-yl,6-(2-carboxy-3,4,5,6-tetrachlorophenyl)-1,11-diethyl-1,2,3,4,8,9,10,11-octa-hydro-2,2,4,8,10,10-hexamethyl perchlorate derivatives may be used as rhodamine dyes.

Cyanine dyes are synthetic dyes having a formula R₂N[CH═CH]ₙCH═N⁺R₂R₂<->R₂N⁺═CH[CH=CH]ₙNR₂ (n is a small numeral). In this formula, nitrogen and conjugated chain moieties usually constitute the part of heterocyclic system such as imidazole, pyridine, pyrrole, quinoline and thiazole. Such dyes are disclosed, for example, in the specifications of U.S. Pat. Nos. 5,268,486, 5,569,587, 5,556,959 and 5,808,044. In certain aspects 1-[3-(4-monomethoxytrityloxy)propyl]-1′-[3-[(2-cyanoethyl)-(N,N-diisopropyl)phosphoramidityl]propyl]-3,3,3′,3′-tetra-methyl-4,5-benzindocarbocyanine (Cy3.5 phosphoramidite) maybe used as a cyanine dye.

The present invention also provides a method for the simultaneous determination of CK19 mRNA and CK20 mRNA, comprising
(a) amplifying a part of the mRNAs in the sample using a pair of primers for CK19 and a pair of primers for CK20 to produce an amplicon from each mRNA, and
(b) detecting the amplicons, using a set of probes for CK19 and a set of probes for CK20;
   wherein the primers for the CK19 mRNA comprise, at least
a first primer capable of hybridizing to a region located on a first exon of the CK19 gene, and
a second primer capable of hybridizing to a region located on a second exon of the CK19 gene located downstream to the first exon;
   and wherein the probes for the CK19 mRNA comprise, at least
a first probe capable of hybridizing to a region located at the 3′-terminus of an exon of the CK19 gene downstream to the region to which the first primer binds and upstream to the region to which the second primer binds, and
a second probe capable of hybridizing to a region located at the 5′-terminus of an adjacent exon of the CK19 gene downstream to the region to which the first probe binds and upstream to the region to which the second primer binds;
   and wherein the primers for the CK20 mRNA comprise, at least
a third primer capable of hybridizing to a region located on the CK20 gene, and
a fourth primer capable of hybridizing to a region located on the CK20 gene downstream of the third primer;
   and wherein the probes for the CK20 mRNA comprise, at least
a third probe capable of hybridizing to a region located on the CK20 gene downstream to the region to which the third primer binds and upstream to the region to which the fourth primer binds, and
a fourth probe capable of hybridizing to a region located on the CK20 gene upstream to the region to which said fourth primer binds and downstream to the region to which said third probe binds.

In certain embodiments of the above-mentioned method, probes for the detection of the CK19 mRNA are labeled with a donor dye and an acceptor dye, probes for the detection of the CK20 mRNA are labeled with a donor dye and an acceptor dye, and, here, the CK19 donor/acceptor dye pair is different from the CK20 donor/acceptor dye pair. In other embodiments the pair of probes for the detection of the CK19 mRNA and the pair of probes for the detection of the CK20 mRNA are labeled with the same donor dye while the acceptor dye for the detection of the CK19 mRNA is different from the acceptor dye for the detection of the CK20 mRNA.

In particular embodiments the first probe and third probe are labeled with donors dyes at their 3′-terminus while the second probe and fourth probe are labeled with acceptor dyes at their 5′-terminus. For example, the donor dye maybe FITC and the acceptor dye may be selected from the group consisting of rhodamine dyes and cyanine dyes. In a specific example, the donor dye is FITC and the acceptor dyes are LC-Red 640 and LC-Red 610, which are rhodamine dyes.

In some embodiments, the amplification of a part of the CK19 mRNA and CK20 mRNA and the detection of the amplified products are carried out in the same reaction vessel.

The present invention further provides a composition of matter useful for the detection of CK19 mRNA, comprising
a first primer capable of hybridizing to a region located on a first exon of the CK19 gene,
a second primer capable of hybridizing to a region located on a second exon of the CK19 gene located downstream to the first exon,
a first probe capable of hybridizing to a region located at the 3′-terminus of an exon of the CK19 gene downstream to the region to which the first primer binds and upstream to the region to which the second primer binds, and
a second probe capable of hybridizing to a region located at the 5′-terminus of an adjacent exon of the CK19 gene downstream to the region to which the first probe binds and upstream to the region to which the second primer binds.

The present invention also provides a primer comprising 10 to 25 base pairs from SEQ ID No. 1 hybridizing to a region located on exon 4 of the CK19 gene, which is the first exon. In certain embodiments the first primer is characterized as being selected from a part of SEQ ID No. 1 containing at least two mismatches to the pseudogene. In some embodiments, the first primer has the sequence of SEQ ID No. 2.

The present invention also provides a primer comprising 10-25 base pairs from Seq. ID No. 3 hybridizing to a region located on a second exon of the CK19 gene downstream to exon 4. In certain embodiments the above-mentioned primer contains at least two mismatches to the CK19 pseudogene. In a particular embodiment the second exon of the CK19 gene is exon 6. In some embodiments, the second primer has the same sequence of SEQ ID No. 4.

The present invention also provides a combination of primers comprising at least one primer as described above as useful for the amplification of a part of the CK19 mRNA. In some embodiments the at least one primer sequence contains at least one mismatch to the CK19 pseudogene at its 3′-terminus.

The present invention also provides a first probe comprising 10 to 25 base pairs from SEQ ID No. 5, which is the complete sequence of CK19 mRNA, hybridizing to a region located at the 3′-terminus of a first exon of the CK19 gene. In certain embodiments the first probe hybridizes to an amplicon prepared by the above-mentioned primer combination. In certain aspects the first exon of the CK19 gene is exon 4. In some embodiments, the sequence of the first probe is SEQ ID No. 6.

The present invention further provides a second probe comprising 10 to 25 base pairs from SEQ ID No. 5, which is the complete sequence of CK19 mRNA, hybridizing to a region located at the 5′-terminus of a second exon of the CK19 gene. In certain embodiments the second probe hybridizes to an exon located downstream of the above-mentioned first probe and being adjacent on an amplicon prepared by the above-mentioned primer combination. In certain aspects the exon located downstream of the first exon of the CK19 gene is exon 5. In some embodiments, the sequence of the second probe is SEQ ID No. 7.

The present invention also relates to combinations of the above-mentioned probes useful for identification of a CK19 mRNA amplicon. The probes may be labeled with a donor dye and an acceptor dye. In certain aspects of the combination of probes the donor dye is an fluorescein dye, such as FITC and the acceptor dye is selected from the group consisting of rhodamine dyes and cyanine dyes. In another embodiment of the combination of probes the first probe is labeled with a donor dye at its 3′-terminus and the second probe bonding in an adjacent manner downstream to the first probe is labeled with an acceptor dye at its 5′-terminus. In that case, for example, the donor dye is FITC and the acceptor dye is a rhodamine dye, e.g., LC-Red 640.

The present invention also provides a kit for the amplification and detection of CK19 mRNA, comprising
a pair of primers, comprising
   a first primer capable of hybridizing to a region located on a first exon of the CK19 gene, and
   a second primer capable of hybridizing to a region located on a second exon of the CK19 gene located downstream to the first exon;
a pair of probes, comprising
   a first probe capable of hybridizing to a region located at the 3′-terminus of an exon of the CK19 gene downstream to the region to which the first primer binds and upstream to the region to which the second primer binds, and
   a second probe capable of hybridizing to a region located at the 5′-terminus of an adjacent exon of the CK19 gene downstream to the region to which the first probe binds and upstream to the region to which the second primer binds.

In certain aspects of the kit the primer pair is present as a mixture or the probe pair is present as a mixture or both the primer pair and the probe pair are present as a mixture. The kit may further contain a buffer solution.

The present invention also provides a kit for the combined amplification and detection of CK19 mRNA and CK20 mRNA. Such a kit at least comprises two pairs of primers and two pairs of probes, wherein the probes are labeled differently for CK19 and CK20. In certain embodiments of the kit, said primers and probes for the amplification and the detection of CK19 mRNA and CK20 mRNA comprise
a first primer capable of hybridizing to a region located on a first exon of the CK19 gene, and
a second primer capable of hybridizing to a region located on a second exon of the CK19 gene located downstream to the first exon;
   and
a first probe capable of hybridizing to a region located at the 3′-terminus of an exon of the CK19 gene downstream to the region to which the first primer binds and upstream to the region to which the second primer binds, and
a second probe capable of hybridizing to a region located at the 5′-terminus of an adjacent exon of the CK19 gene downstream to the region to which the first probe binds and upstream to the region to which the second primer binds,
   and
a third primer capable of hybridizing to a region located on the CK20 gene, and
a fourth primer capable of hybridizing to a region located on the CK20 gene downstream of the third primer;
   and
a third probe capable of hybridizing to a region located on the CK20 gene downstream to the region to which the third primer binds and upstream to the region to which the fourth primer binds, and
a fourth probe capable of hybridizing to a region located on the CK20 gene upstream to the region to which the fourth primer binds and downstream to the region to which the third probe binds.

In certain aspects of the above-mentioned kit, the primer pair is present as a mixture or the probe pair is present as a mixture or both the primer pair and the probe pair are present as a mixture. The kit may in certain embodiments further contain a buffer solution.

The present invention provides novel PCR primers and probes for the specific amplification and detection of CK19 mRNA by real-time RT-PCR and hybridization probe techniques, particularly useful for clinical testing. Furthermore, the reaction conditions therefore are provided.

In order to achieve an RT-PCR which is highly specific to CK19 mRNA even when human DNA is contaminating a sample, the region and positions of PCR primers and detection probes were determined on the basis of the following designs:

1) A design is carried out so that, in RT-PCR, each 3′-terminus of the two PCR primers comprises mismatch sites of the pseudogene of CK19 and CK19 mRNA whereby amplification of the CK19 pseudogene is not possible even when amplification of CK 19 mRNA is possible.

2) With regard to the region to be subjected to gene amplification, the site which satisfies the above-mentioned condition and which contains at least one boundary of exon-intron of the CK19 gene is selected.

3) When two probes for detection hybridize to a target sequence on the basis of a hybridization probe technique, the design is done in such a manner that the aforementioned exon-intron boundary is located between the hybridizing positions of the two probes for detection. The design may in other cases also be done in such a manner that one of the two probes for detection hybridizes onto an exon-intron boundary whereby an amplicon derived from the CK19 gene is not identified.

1) Design of PCR Primer

The PCR primers provided by the present invention complementarily hybridize to CK19 mRNA. If the PCR primers could also hybridize to the CK19 pseudogene, the setting is deferred to a base region where the 3′-terminus of the PCR primers is not complementary to at least one base or preferably two bases.

Since the present invention is applied to clinical diagnosis, PCR primers are designed so as to make a real-time PCR possible. To be more specific, the size of the PCR primers should be 10 to 25 bases or 17 to 18 bases. With regard to GC %, it may be 45% to 55%. The melting temperature, Tm, may be 45° C. to 55° C. or around 52° C.

In order to shorten the PCR amplifying time, the size of the amplicon amplified by the PCR may be 150 to 250 base pairs. In addition, one or more exon-intron boundary/boundaries of the gene may be present in the region which is amplified by the PCR.

In a region which is amplified by the PCR, a site to which a probe for detection hybridizes should be present.

2) Design of Probes for Detection

With regard to the two kinds of probes for detection provided by the present invention, each of them complimentarily hybridizes to CK19 mRNA amplified by the PCR.

Two kinds of probes for detection are DNA oligonudeotides labeled with different fluorescent dyes and one is called a donor probe while another is called an acceptor probe. In the donor probe, the 5′-terminus is labeled with a fluorescent dye while, in the acceptor probe, the 3′-terminus is subjected to a fluorescent labeling.

Those two probes for detection are designed so as to hybridize to very near regions which are separated by only 1 to 5 base(s). As a result thereof, the so-called fluorescence resonance energy transfer (FRET) occurs between fluorescent dyes labeled on two probes. To be more specific, when light which excites the fluorescent dye of donor probe is irradiated, the excited energy transfers to the fluorescent dye on the adjacent acceptor probe whereupon fluorescence of that dye is generated. Such a phenomenon occurs only when the two probes hybridize to the closely adjacent sites.

The size of the probe for detection may be between 10 to 25 bases or 18 to 22 bases. GC % may be 45% to 55%. The melting temperature should be 50° C. to 65° C. and is set to be 5° C. higher than the melting temperature of the primers and, furthermore, the melting temperature of the acceptor probe is set to be 2 to 3° C. higher than the melting temperature of the donor probe.

3) RT-PCR Method and Reaction Protocol

The PCR using the PCR primers and detection probes provided by the present invention may be carried out in a thermal cycler where all real-time PCRs are possible. At that time, the temperature control property of each machine and the corresponding fluorescence filter are different and, therefore, upon necessity, optimization of the PCR conditions and the selection of fluorescent dyes are required. Here in the Examples, the PCR conditions will be illustrated when a Light Cycler® is used (Roche Diagnostic K. K., Minato-Ku, Tokyo, Japan.).

In carrying out the RT-PCR, mRNA which is the target is first subjected to a reverse transcription to synthesize cDNA and then the PCR is conducted. The PCR using the PCR primers and detection probes provided by the present invention may be used when the reverse transcription step and the PCR step are separately performed (two-step RT-PCR). In other applications it is also possible to use the primers in case where the reverse transcription step and the PCR step are conducted in one reaction vessel (one-step RT-PCR). Here in the Examples, the result of a one-step RT-PCR which is quicker is shown.

4) Method for Detection of PCR Amplicon

In order to use the detection probes described therein, it is necessary to use a thermal cycler which is equipped with a light source generating a wavelength which is able to excite the fluorescent dye of the donor probes and which is also equipped with a detector able to measure the fluorescence of the fluorescent dye of the acceptor probes.

Here in Examples, the PCR conditions are shown when a Light Cycler® (distributor: Roche Diagnostic K. K.) is used.

5) Reagent and Reagent Kit

The PCR primers and the detection probes of the present invention may also be provided in a kit together with reagents which are necessary for amplification and detection of mRNA of the CK19 gene as the target.

### EXAMPLES

Examples of the present invention will be shown as follows although the present invention is not limited to those Examples only.

### Example 1

### Design of PCR Primers

As mentioned already, the pseudogene of a process type has been report for CK19. It is necessary to prepare PCR primers which are not affected even when such a pseudogene is contaminating the sample.

For such a purpose, three kinds of forward primers (F1 to F3) and two kinds of reverse primers (R1 to R2) were prepared. F1 to F3 and R1 were designed in such a manner that their 3′-terminus exhibit a mismatch site of the CK19 pseudogene and the CK19 mRNA. R2 was designed in such a manner that a boundary of intron-exon is present in its base sequence. F1 and R1 contain two mismatches to the CK19 pseudogene and F2, F3 and R2 have one mismatch to the CK19 pseudogene.

(SEQ ID No. 2)

F1: TGAGTGACATGCGAAGC
(799 to 815 of bases of SEQ ID No. 5)

(SEQ ID No. 8)

F2: CGCCAAGATCCTGAGTG
(788 to 804 of bases of SEQ ID No. 5)

(SEQ ID No. 9)

F3: GACATGCGAAGCCAATAT
(804 to 821 of bases of SEQ ID No. 5)

(SEQ ID No. 4)

R1: TGTGTCTTCCAAGGCA
(1007 to 1022 of bases of SEQ ID No. 5)

(SEQ ID No. 10)

R2: CCAAGGCAGCTTTCAT
(999 to 1014 of bases of SEQ ID No. 5)

PCR was carried out under the following conditions using primer combinations of F1/R1, F1/R2, F2/R2 and F3/R2.

With regard to an enzyme used for the reaction (Tth DNA polymerase), a commercially available kit (Light Cycler® RNA Master Hybridization Probes; distributor: Roche Diagnostic K. K.) was used.

| | |
|---|---|
| 50 mM manganese acetate | 3.25 mM |
| PCR primers | 0.25 μM each |
| Tth DNA polymerase | 7.5 μl/reaction |
| CK19 mRNA or CK19 pseudogene | 10⁵ copies/PCR |

A reaction solution (20 μl) containing the above components was placed in a glass capillary and subjected to a one-step RT-PCR using a Light Cycler® under the condition as shown in Table 1 and 5 μl of the reaction product were analyzed by 3% agarose gel electrophoresis.

TABLE 1

| | |
|---|---|
| Cycle Program Data | Value |

Program 1: RT

| | |
|---|---|
| Cycles | 1 |
| Analysis Mode | None |
| Temperature Targets | Segment 1 |
| Target Temperature (° C.) | 61 |
| Incubation time (h:min:s) | 00:05:00 |
| Temperature Transition Rate (° C./s) | 20.0 |
| Secondary Target Temperature (° C.) | 0 |
| Step Size (° C.) | 0.0 |
| Step Delay (Cycles) | 0 |
| Acquisition Mode | None |

Program 2: Denaturation

| | |
|---|---|
| Cycles | 1 |
| Analysis Mode | None |
| Temperature Targets | Segment 1 |
| Target Temperature (° C.) | 95 |
| Incubation time (h:min:s) | 00:00:30 |
| Temperature Transition Rate (° C./s) | 20.0 |
| Secondary Target Temperature (° C.) | 0 |
| Step Size (° C.) | 0.0 |
| Step Delay (Cycles) | 0 |
| Acquisition Mode | None |

Program 3: Amplification

| Cycles | 40 | | |
|---|---|---|---|
| Analysis Mode | None | | |
| Temperature Targets | Segment 1 | Segment 2 | Segment 3 |
| Target Temperature (° C.) | 95 | 45 | 72 |
| Incubation time (h:min:s) | 00:00:01 | 00:00:15 | 00:00:05 |
| Temperature Transition Rate (° C./s) | 20.0 | 20.0 | 2.0 |
| Secondary Target Temperature (° C.) | 0 | 0 | 0 |
| Step Size (° C.) | 0.0 | 0.0 | 0.0 |
| Step Delay (Cycles) | 0 | 0 | 0 |
| Acquisition Mode | None | Single | None |

Program 4: Cooling

| | |
|---|---|
| Cycles | 1 |
| Analysis Mode | None |
| Temperature Targets | Segment 1 |
| Target Temperature (° C.) | 40 |
| Incubation time (h:min:s) | 00:00:30 |
| Temperature Transition Rate (° C./s) | 20.0 |
| Secondary Target Temperature (° C.) | 0 |
| Step Size (° C.) | 0.0 |
| Step Delay (Cycles) | 0 |
| Acquisition Mode | None |

In F1/R1 (in both F1 and R1, two mismatches to the CK19 pseudogene are present), although some bands supposed to be amplicon from the CK19 gene were noted, no amplicon from CK19 pseudogene was noted at all.

In F1/R2, F2/R2 and F3/R2 (in all of R2, F2 and F3, one mismatch to CK19 pseudogene is present), although no band of an amplicon from the CK19 gene were noted, a small amount of amplicon from CK19 pseudogene was noted.

It was judged to be difficult to avoid amplification of the CK19 pseudogene by further improvement in the detection probes. Therefore the F1/R1 primer pair, where no amplificate from CK19 pseudogene was noted, was chosen to be used further.

### Example 2

### Design of Detection Probes

Two sets of detection probes were prepared and compared. With regard to donor probes (P1 and P1b), their 3′-terminus was labeled with FITC while, with regard to acceptor probes, their 5′-terminus was labeled with LC-Red 460.

Set 1

(SEQ ID No. 11)

P1: GTCATGGCCGAGCAGAACC
(825 to 843 of bases of SEQ ID No. 5)

(SEQ ID No. 12)

P2: AAGGATGCTGAAGCCTGGT
(846 to 864 of bases of SEQ ID No. 5)
Set 2

(SEQ ID No. 6)

P1b: AAGCCTGGTTCACCAGCCG
(856 to 874 of bases of SEQ ID No. 5)

(SEQ ID No. 7)

P2c: CTGAAGAATTGAACCGGGAGG
(877 to 897 of bases of SEQ ID No. 5)

Set 2 was designed in such a manner that a boundary of exon-intron is located between the hybridization positions of the two probes while set 1 was not designed as such.

For evaluation of probe sets, 20 μl of a reaction solution containing the following components was placed in a glass capillary and subjected to a one-step RT-PCR using a Light Cycler® under the condition as shown in Table 1.

| | |
|---|---|
| 50 mM manganese acetate | 3.25 mM |
| PCR primers F1 and R1 | 0.25 μM each |
| Donor probe (P1 or P1b) | 25 nM |
| Acceptor probe (P1 or P2c) | 100 nM |
| Tth DNA polymerase | 7.5 μl/reaction |
| CK19 mRNA or CK19 pseudogene | 10⁵ copies/PCR |

As a result, fluorescent signal due to amplification of the CK19 gene was noted in set 1 while, in set 2, no fluorescent signal was noted at all.

From the above investigations, there was established a combination of primers (F1 and R1) and probes (P1b and P2c) where amplification of the CK19 pseudogene was excluded by selection of the PCR primers and amplification signal of the CK19 gene was excluded by selection of the detection probes.

### Example 3

### Real-Time RT-PCR of CK19 mRNA

An example of a real-time RT-PCR of CK19 mRNA which is a positive control and human DNA and CK19 pseudogene is shown.

CK19 mRNA was diluted 10-fold in a stepwise manner and 10⁵ to 10² copies were subjected to a one-step RT-PCR amplification using a Light Cycler®. Similarly, 500 ng of human DNA or 10⁵ copies of CK19 pseudogene were amplified and detected by RT-PCR (time required was about 40 minutes). The compositions of the reaction solution (20 μl/PCR) were as follows below.

| | |
|---|---|
| 50 mM manganese acetate | 3.25 mM |
| PCR primers F1 and R1 | 0.25 μM each |
| Donor probe P1b | 25 nM |
| Acceptor probe P2c | 100 nM |
| Tth DNA polymerase | 7.5 μl/reaction |

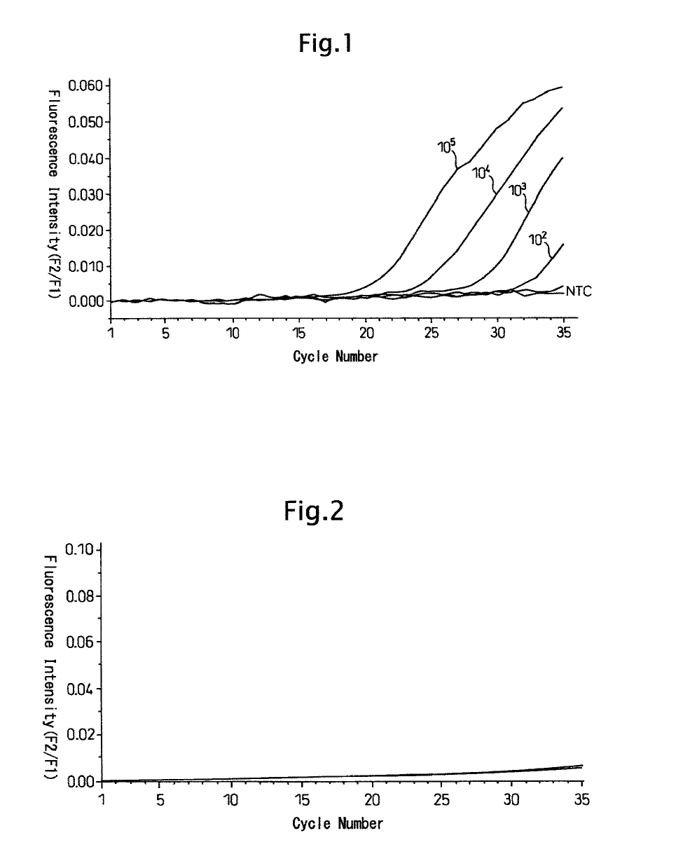

The results are shown in FIG. 1 and FIG. 2. In those figures, the ordinate shows intensity of fluorescence while the abscissa shows PCR cycle numbers.

When CK19 mRNA was amplified by RT-PCR, fluorescent signals were generated in PCR cycle numbers depending upon the initial amount (FIG. 1) while, when human DNA or CK19 pseudogene was subjected to PCR similarly, no such fluorescent signal was noted (FIG. 2).

### Example 4

### Detection of CK19 mRNA in Human Lymph Node

An example of real-time RT-PCR of CK19 mRNA using a clinical sample is shown. Among lymph nodes excised from a patient suffering from stomach cancer of cT INO, the lymph nodes where metastasis of cancer was noted by a pathological diagnosis and the lymph nodes where no metastasis of cancer was noted were selected and homogenized in a buffer solution (600 μl) containing guanidine thiocyanate. The homogenization was carried out using a MagNA Lyser® (distributor: Roche Diagnostic K. K.). Distilled water (500 μl) was added to 450 μl of the homogenate and centrifuged at 14500×g and the supernatant liquid (900 μl) was transferred to a new tube. To this 2 μl of an oligonucleotide (biotin-5′-GCTTCACATCCCTCCGCTGATTCTCTTGA) labeled with biotin was added and the mixture was allowed to stand at 37° C. for 10 minutes whereupon the oligonucleotide and CK19 mRNA hybridized.

After that, magnetic particles coated with avidin were added thereto and the mixture was allowed to stand at 37° C. for 10 minutes more whereupon an mRNA-oligonucleotide complex was trapped on the magnetic particles. The magnetic particles were washed with a buffer solution containing a surfactant twice to remove excess components, then 50 μl of a TE buffer solution was added so that the magnetic particles were well suspended therein and CK19 mRNA on the magnetic particles was extracted with heat (time required was about 45 minutes).

The extracted sample (2 μl) was subjected to a one-step RT-PCR. The specific conditions were the same as in Example 3 (time required was about 40 minutes).

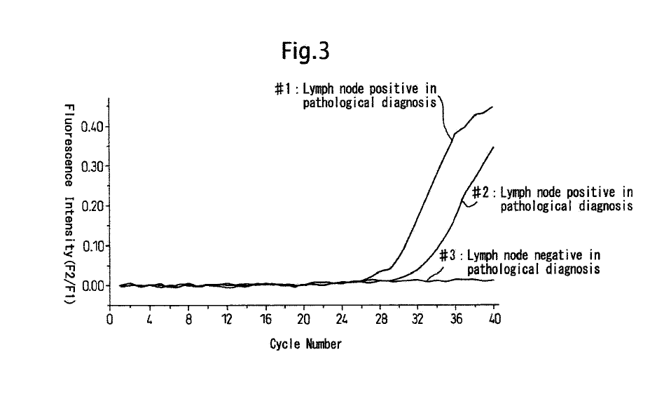

The result is shown in FIG. 3. The lymph nodes (#1 to #2) where metastasis of cancer was positive in the pathological diagnosis were positive in the RT-PCR while the lymph node (#3) where metastasis of cancer was negative in the pathological diagnosis was negative in the RT-PCR.

### Example 5

### An Example of Multiplex Real-Time RT-PCR

In clinical tests, plural test items are sometimes tested by one measurement. This is important for saving costs and labor. Especially when diagnosis for presence or absence of cancer cells and for metastasis is conducted, judgment by the result of one single marker only has a certain possibility of risk of a wrong diagnosis. Accordingly, if other markers in addition to CK19 mRNA may be subjected to a real-time RT-PCR at the same time, the usefulness is enhanced in applying the test to clinical diagnosis.

In the example, mRNA of CK20 which is the same kind of cytokeratin as CK19 was subjected to amplification together with CK19 mRNA. Furthermore, a protocol for a separate detection was established. Although the conditions for RT-PCR were the same as that in the Example 3, PCR primers (CK20F and CK20R) and detection primers (CK20P1 and CK20P2) for CK20 were added to the reaction solution. In the detection probes, 3′-terminus was labeled with FITC for the donor probes while, for acceptor probes, 5′-terminus was labeled with LC-Red 610 for discriminating from the detection wavelength of CK19.

| | |
|---|---|
| CK20F: | |
| ATCAAGCAGTGGTACGAAAC | (SEQ ID No. 13) |
| CK20R: | |
| AGGACACACCGAGCATTT | (SEQ ID No. 14) |
| CK20P1: | |
| ATTACAGACAAATTGAAGAGCTGCC | (SEQ ID No. 15) |
| CK20P2: | |
| AGTCAGATTAAGGATGCTCAACTGC | (SEQ ID No. 16) |

| | |
|---|---|
| 50 mM manganese acetate | 3.25 mM |
| CK19 PCR primers F1 and R1 | 0.25 μM each |
| CK19 donor probe P1b | 25 nM |
| CK19 acceptor probe P2c | 100 nM |
| CK20 PCR primers CK20F and CK20R | 0.25 μM each |
| CK20 donor probe CK20P1 | 25 nM |
| CK20 acceptor probe CK20P2 | 100 nM |
| Tth DNA polymerase | 7.5 μl/reaction |
| Positive control, mRNA of CK19 gene | |
| Positive control, mRNA of CK20 gene | |

Both mRNA for CK19 and CK20 were amplified in the same tube and their amplifications were separately monitored due to their different detection wavelength (640 nm for CK19 and 610 nm for CK20).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method for the detection of CK19 mRNA in a sample, comprising:
(c) amplifying a part of the mRNA in said sample using a pair of primers, to produce an amplicon, and
(d) detecting said amplicon using two probes;
wherein said primers comprise, at least
a first primer capable of hybridizing to a 1^{st} region located on a first exon of the CK19 gene, and
a second primer capable of hybridizing to a 2^{nd} region located on a second exon of the CK19 gene located downstream from the first exon; and
wherein said probes comprise, at least
a first probe capable of hybridizing to a 3^{rd} region located at the 3′-terminus of a first exon of the CK19 gene, said region being located downstream of the region to which said first primer binds and upstream of the region to which said second primer binds, and
a second probe capable of hybridizing to a 4^{th} region located at the 5′-terminus of a second exon of the CK19 gene, said second exon being adjacent to said 1^{st} exon and being downstream to the 4^{th} region to which said first probe binds and upstream to the region to which said second primer binds.

2. The method according to claim 1, wherein the probes are labeled with a donor dye and an acceptor dye.

3. The method according to claim 2, wherein the donor is a fluorescein dye and the acceptor is selected from a group consisting of rhodamine dyes and cyanine dyes.

4. The method according to claim 2, wherein the first probe is labeled with a donor at its 3′-terminus and wherein the second probe is labeled with an acceptor at its 5′-terminus.

5. The method according to claim 1, wherein any one of said primers comprises at least two mismatches to the CK19 pseudogene.

6. A method for the simultaneous determination of CK19 mRNA and CK20 mRNA in a sample, comprising:
(c) amplifying a part of the mRNAs in said sample using a pair of primers for CK19 and a pair of primers for CK20 to produce an amplicon from each mRNA, and
(d) detecting said amplicons, using a set of probes for CK19 and a set of probes for CK20;
wherein said primers for the CK19 mRNA comprise, at least
a first primer capable of hybridizing to a region located on a first exon of the CK19 gene, and
a second primer capable of hybridizing to a region located on a second exon of the CK19 gene located downstream to the first exon;
and wherein said probes for the CK19 mRNA comprise, at least
a first probe capable of hybridizing to a region located at the 3′-terminus of an exon of the CK19 gene downstream to the region to which said first primer binds and upstream to the region to which said second primer binds, and
a second probe capable of hybridizing to a region located at the 5′-terminus of an adjacent exon of the CK19 gene downstream to the region to which said first probe binds and upstream to the region to which said second primer binds;
and wherein said primers for the CK20 mRNA comprise, at least
a third primer capable of hybridizing to a region located on the CK20 gene, and
a fourth primer capable of hybridizing to a region located on the CK20 gene downstream of the third primer;
and wherein said probes for the CK20 mRNA comprise, at least
a third probe capable of hybridizing to a region located on the; CK20 gene downstream to the region to which said third primer binds and upstream to the region to which said fourth primer binds, and
a fourth probe capable of hybridizing to a region located on the CK20 gene upstream to the region to which said fourth primer binds and downstream to the region to which said third probe binds.

7. The method according to claim 6, wherein each of the probes for the detection of CK19 and the probes for the detection of CK20 are labeled with a donor dye and an acceptor dye, wherein the CK19 donor dye/acceptor dye pair differs from the CK20 donor dye/acceptor dye pair.

8. The method according to claim 6, wherein the probes for the detection of CK19 and of probes for the detection of CK20 contain the same donor dye, and the acceptor dye on the probes for the detection of CK19 differs from the acceptor dye on the probes for the detection of CK20.

9. The method according to claim 7, wherein the first and the third probe are labeled with a donor dye at their 3′-terminus and wherein the second and the fourth probe are labeled with an acceptor dye at their 5′-terminus.

10. The method according to claim 7, wherein the donor dye is a fluorescein dye and the acceptor dye is selected from a group consisting of rhodamine dyes and cyanine dyes.

11. The method according to claim 6, wherein any one of said primers for the CK19 mRNA comprises at least two mismatches to the sequence of the CK19 pseudogene.

12. A kit for the amplification and detection of CK19 mRNA, comprising:
a pair of primers, comprising
a first primer capable of hybridizing to a region located on a first exon of the CK19 gene, and
a second primer capable of hybridizing to a region located on a second exon of the CK19 gene located downstream to said first exon;
a pair of probes, comprising
a first probe capable of hybridizing to a region located at the 3′-terminus of an exon of the CK19 gene downstream to the region to which said first primer binds and upstream to the region to which said second primer binds, and
a second probe capable of hybridizing to a region located at the 5′-terminus of an adjacent exon of the CK19 gene downstream to the region to which said first probe binds and upstream to the region to which said second primer binds.

13. The kit according to claim 12, wherein the pair of primers is contained in a mixture or wherein the pair of probes is contained in a mixture or wherein the pair of primers and the pair of probes are contained in a mixture.

14. The kit according to claim 12, further containing a buffer solution.

15. A kit for the combined amplification and detection of CK19 mRNA and CK20 mRNA, comprising:
a first primer capable of hybridizing to a region located on a first exon of the CK19 gene, and
a second primer capable of hybridizing to a region located on a second exon of the CK19 gene located downstream to said first exon; and
a first probe capable of hybridizing to a region located at the 3′-terminus of an exon of the CK19 gene downstream to the region to which said first primer binds and upstream to the region to which said second primer binds, and
a second probe capable of hybridizing to a region located at the 5′-terminus of an adjacent exon of the CK19 gene downstream to the region to which said first probe binds and upstream to the region to which said second primer binds, and
a third primer capable of hybridizing to a region located on the CK20 gene, and
a fourth primer capable of hybridizing to a region located on the CK20 gene downstream of the third primer; and
a third probe capable of hybridizing to a region located on the CK20 gene downstream to the region to which said third primer binds and upstream to the region to which said fourth primer binds, and
a fourth probe capable of hybridizing to a region located on the CK20 gene upstream to the region to which said fourth primer binds and downstream to the region to which said third probe binds.

16. The kit according to claim 15, wherein primers are contained in a mixture or the probes are contained in a mixture or the primers and the probes are contained in a mixture.

17. The kit according to claim 15, further containing a buffer solution.

18. A reaction mixture, comprising:
a first primer capable of hybridizing to a region located on a first exon of the CK19 gene,
a second primer capable of hybridizing to a region located on a second exon of the CK19 gene located downstream to said first exon,
a first probe capable of hybridizing to a region located at the 3′-terminus of an exon of the CK19 gene downstream to the region to which said first primer binds and upstream to the region to which said second primer binds, and
a second probe capable of hybridizing to a region located at the 5′-terminus of an adjacent exon of the CK19 gene downstream to the region to which said first probe binds and upstream to the region to which said second primer binds.

19. The reaction mixture according to claim 19, wherein any of said primers comprises at least two mismatches to the CK19 pseudogene.


SEQUENCE LISTING (16 sequences)

SEQ ID NO: 1 - DNA, 115 bp, Homo sapiens
  Part of mRNA encoding cytokeratin 19
ggtggattccgctccgggcaccgatctcgccaagatcctgagtgacatgcgaagccaatatgaggtcatggccgagcagaaccggaaggatgctgaagcctggttcaccagccgg

SEQ ID NO: 2 - DNA, 17 bp, Artificial Sequence
  Primer F1
tgagtgacatgcgaagc

SEQ ID NO: 3 - DNA, 559 bp, Homo sapiens
  Part of mRNA encoding cytokeratin 19
gatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaagggaaaaaaaaaaaaaaaaaaaaaaaaaa

SEQ ID NO: 4 - DNA, 16 bp, Artificial Sequence
  Primer R1
tgtgtcttccaaggca

SEQ ID NO: 5 - DNA, 1407 bp, Homo apiens
  Exon 1 1..473
  Exon 2 374..556
  Exon 3 557..713
  Exon 4 714..875
  Exon 5 876..1001
  Exon 6 1002..1380
cgcgaatcgcagcttctgagaccagggttgctccgtccgtgctccgcctcgccatgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaagggaaaaaaaaaaaaaaaaaaaaaaaaaa

SEQ ID NO: 6 - DNA, 19 bp, Artificial Sequence
  Probe P1b
aagcctggttcaccagccg

SEQ ID NO: 7 - DNA, 21 bp, Artificial Sequence
  Probe P2c
ctgaagaattgaaccgggagg

SEQ ID NO: 8 - DNA, 17 bp, Artificial Sequence
  Primer F2
cgccaagatcctgagtg

SEQ ID NO: 9 - DNA, 18 bp, Artificial Sequence
  Primer F3
gacatgcgaagccaatat

SEQ ID NO: 10 - DNA, 16 bp, Artificial Sequence
  Primer R2
ccaaggcagctttcat

SEQ ID NO: 11 - DNA, 19 bp, Artificial Sequence
  Probe P1
gtcatggccgagcagaacc

SEQ ID NO: 12 - DNA, 19 bp, Artificial Sequence
  Primer F1
aaggatgctgaagcctggt

SEQ ID NO: 13 - DNA, 20 bp, Artificial Sequence
  20: Primer CK20F
atcaagcagtggtacgaaac

SEQ ID NO: 14 - DNA, 18 bp, Artificial Sequence
  Primer CK20R
aggacacaccgagcattt

SEQ ID NO: 15 - DNA, 25 bp, Artificial Sequence
  Probe CK20P1
attacagacaaattgaagagctgcc

SEQ ID NO: 16 - DNA, 25 bp, Artificial Sequence
  Primer CK20P2
agtcagattaaggatgctcaactgc